(19) United States
(12) Patent Application Publication
(10) Pub. No.: US 2026/0363811 A1
(43) Pub. Date: Nov. 19, 2026

(54) CONTROL SYSTEM FOR A SURGICAL ROBOTIC SYSTEM

(71) Applicant: CMR Surgical Limited, Cambridge (GB)

(72) Inventors: Edward James Wildin Tucker, Cambridge (GB); Isabelle Ascah-Coallier, Cambridge (GB); Johnathan Man Chiu Hung, Cambridge (GB); Fiona Middlemiss Haig, Cambridge (GB); Pedro Angel Riera Martinez, London (GB); Andrew Robert Mawby, London (GB)

(73) Assignee: CMR Surgical Limited, Cambridge, OT (GB)

(21) Appl. No.: 19/135,670

(22) PCT Filed: Dec. 6, 2023
(86) PCT No.: PCT/GB2023/053152

§ 371 Date: Jun. 4, 2025

(30) Foreign Application Priority Data
Dec. 6, 2022 (GB) 2218329.7

(51) Int. Cl. A61B 34/37; A61B 17/00; A61B 34/00; A61B 34/30; A61B 90/00

(52) U.S. Cl. CPC A61B 34/37; A61B 17/00; A61B 34/25; A61B 34/77; A61B 90/361; A61B 2017/00203; A61B 2034/254; A61B 2034/306

(57) ABSTRACT

A control system and method for a surgical robotic system having a surgical robot arm In response to identifying A condition is identified that suggests rotation of a surgical instrument attached to the surgical arm. A rotational scaling mode is enabled in response to receiving a mode selection input. A received control input is used to determine an indication of a movement of the surgeon input device which includes a matrix representation of a rotation of the surgeon input device. The matrix representation is converted into an axis-angle representation including an indication of a rotation axis and an angle of rotation about the rotation axis. A scaled angle of rotation is determined for a scaled axis-angle representation of a scaled rotation. A control signal is generated using the scaled axis-angle representation of the scaled rotation and sent to the surgical robot arm in order to drive the surgical robot arm.

### BACKGROUND

This invention relates to a control system for a surgical robotic system.

It is known to use robots for assisting and performing surgery. FIG. 1 illustrates an example surgical robotic system 100, which comprises a surgical robot arm 101 for manipulating tissue. The surgical robot arm 101 comprises a base 109. The base supports the surgical robot arm, and is itself attached rigidly to, for example, the operating theatre floor, the operating theatre ceiling or a trolley. The surgical robot arm 101 is articulated by means of multiple joints 104 along its length, which are used to locate a robotic surgical instrument 106 in a desired location relative to a patient 102. The robotic surgical instrument 106 could, for example, be a cutting or grasping device or a needle holder. A robotic surgical instrument 106 is attached to the distal end of the surgical robot arm 101. During a surgical procedure, the robotic surgical instrument 106 may be inserted into the body of the patient 102 (optionally, via an access port 117) so as to access a surgical site within the body of the patient 102. At its distal end the robotic surgical instrument comprises an end effector for performing aspects of a medical procedure. This type of medical procedure is often referred to as a minimally invasive surgical procedure.

The configuration of the surgical robot arm 101 may be remotely controlled in response to inputs received at a remote surgeon console 120. A surgeon may provide inputs to the surgeon console. The remote surgeon console may comprise one or more surgeon input devices 123. For example, these may take the form of one or more hand controllers, foot pedals, interactive touch screens etc. A video feed of the surgical site may be captured by an endoscope, often attached to a further surgical robot arm (not shown in FIG. 1 for simplicity), and displayed at a display 121 of the remote surgeon console.

A control system 124 connects the surgeon console 120 and the surgical robot arm 101. The control system 124 receives inputs from the surgeon input device(s) 123 and converts those inputs to control signals for controlling the surgical robot arm 101. The conversion of the inputs to the control signals can be performed using inverse kinematics.

The international patent application, published as WO 2017/100434, discloses a robotic surgical system in which an input device is rotatable about a first axis of rotation and a second axis of rotation. A processing unit is operatively associated with a linkage to rotate a surgical tool about a first axis of movement based on a scaled rotation of the input device about the first axis of rotation by a first scaling factor and to rotate the surgical tool about a second axis of movement based on a scaled rotation of the input device about the second axis of rotation by a second scaling factor that is different from the first scaling factor.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

There is provided a control system for a surgical robotic system, the surgical robotic system comprising a surgical robot arm and a surgeon input device, wherein the surgical robot arm comprises: (i) a series of joints by which its configuration can be altered, and (ii) an attachment for a surgical instrument at a distal end of the surgical robot arm, wherein the control system is configured to:
identify a condition that suggests that the surgical robot arm is being used for a surgical step involving rotation of a surgical instrument attached to the surgical robot arm;
in response to identifying said condition, output a prompt for prompting a user to enable a rotational scaling mode;
receive a mode selection input that indicates whether the user has selected to enable the rotational scaling mode; and
enable the rotational scaling mode in response to receiving a mode selection input that indicates that the user has selected to enable the rotational scaling mode;
wherein the control system is configured to, when the rotational scaling mode is enabled:
   receive a control input from the surgeon input device;
   use the received control input to determine an indication of a movement of the surgeon input device, wherein the indication of the movement comprises a matrix representation of a rotation of the surgeon input device;
   convert the matrix representation into an axis-angle representation of the rotation of the surgeon input device, wherein said axis-angle representation of the rotation comprises: (i) an indication of a rotation axis for the rotation, and (ii) an angle of rotation about the rotation axis;
   scale the angle of rotation with a rotational scaling factor to determine a scaled angle of rotation about the rotation axis for a scaled axis-angle representation of a scaled rotation;
   generate a control signal for the surgical robot arm using the scaled axis-angle representation of the scaled rotation; and
   cause the generated control signal to be sent to the surgical robot arm in order to drive the surgical robot arm such that its configuration is altered.

The surgical robotic system may comprise a surgeon console which comprises the surgeon input device, and wherein the control system may be configured to output a prompt for prompting a user to enable a rotational scaling mode by:
causing a visual indication to be displayed at the surgeon console prompting the user to enable the rotational scaling mode, and/or
causing an audio indication to be output from the surgeon console prompting the user to enable the rotational scaling mode.

The control system may be configured to receive the mode selection input responsive to the user making a selection on the surgeon console to enable the rotational scaling mode.

The selection may be made by the user by:
selecting an option displayed on a display of the surgeon console;
issuing a voice command which is received by a microphone of the surgeon console; or
performing a gesture which is identified in images of the user captured by a camera of the surgeon console.

The mode selection input may indicate the rotational scaling factor based on a user selection of the rotational scaling factor.

The control system may be further configured to determine an instrument type of a surgical instrument attached to the surgical robot arm, wherein the control system may be configured to enable the rotational scaling mode and/or scale the angle of rotation in dependence upon the determined instrument type of the surgical instrument.

The control system may be configured to determine the instrument type of the surgical instrument by reading an indication of the instrument type from an instrument memory located on the surgical instrument.

The control system may be configured to enable the rotational scaling mode only if the determined instrument type indicates that the surgical instrument is a needle holder. Said surgical step involving rotation of a surgical instrument attached to the surgical robot arm may be suturing.

The control system may be configured to identify the condition that suggests that the surgical robot arm is being used for suturing by:
determining that the surgical robotic system currently has one or more operational surgical robot arms with needle holder instruments attached thereto;
determining that the surgical robotic system currently has two or more operational surgical robot arms with a predetermined combination of surgical instruments attached thereto, said predetermined combination of surgical instruments being suitable for performing suturing;
determining that the current time in a surgical procedure matches a time at which suturing is planned to be performed in the surgical procedure;
analysing one or more images of a surgical site captured by an endoscope in the surgical robotic system; and/or
determining that a speed and/or pattern of motion of the surgical robot arm matches that expected during suturing.

The control system may be further configured to:
determine that the surgical robot arm cannot alter its configuration to achieve a desired configuration in accordance with the generated control signal using the scaled axis-angle representation of the scaled rotation;
in response to determining that the surgical robot arm cannot alter its configuration to achieve the desired configuration, scale the angle of rotation with an adjusted rotational scaling factor to determine an adjusted scaled angle of rotation about the rotation axis for an adjusted scaled axis-angle representation of an adjusted scaled rotation;
generate an adjusted control signal for the surgical robot arm using the adjusted scaled axis-angle representation of the adjusted scaled rotation; and
cause the adjusted generated control signal to be sent to the surgical robot arm in order to drive the surgical robot arm such that its configuration is altered.

The control system may be configured to generate the control signal for the surgical robot arm using the scaled axis-angle representation of the scaled rotation by:
converting the scaled axis-angle representation of the scaled rotation to a matrix representation of the scaled rotation; and
generating the control signal for the surgical robot arm using the matrix representation of the scaled rotation.

The control system may be configured to disable the rotational scaling mode in response to receiving a mode selection input that indicates that the user has selected to disable the rotational scaling mode, wherein the control system may be configured to, when the rotational scaling mode is disabled:
receive a control input from the surgeon input device;
use the received control input to determine an indication of a movement of the surgeon input device, wherein the indication of the movement comprises a matrix representation of a rotation of the surgeon input device;
generate a non-rotationally scaled control signal for the surgical robot arm using the determined indication of a movement of the surgeon input device without converting the matrix representation into an axis-angle representation of the rotation of the surgeon input device and without scaling the angle of rotation with the rotational scaling factor; and
cause the generated non-rotationally scaled control signal to be sent to the surgical robot arm in order to drive the surgical robot arm.

The control system may be configured to, when the rotational scaling mode is enabled:
identify a further condition that suggests that the surgical robot arm is not being used for a surgical step involving rotation of a surgical instrument attached to the surgical robot arm; and
in response to identifying said further condition, output a prompt for prompting the user to disable the rotational scaling mode.

The surgeon input device may comprise a hand controller connected to a gimbal assembly, and wherein said received control input may comprise indications of joint positions of the gimbal assembly.

The control system may be configured to control the surgical robot arm, in dependence on the control inputs received from the surgeon input device, to alter the configuration of the surgical robot arm whilst maintaining an intersection between a surgical instrument attached to the surgical robot arm and a pivot point.

The indication of the movement of the surgeon input device may further comprise a representation of a translational movement of the surgeon input device, wherein the control system may be configured to:
scale the translational movement of the surgeon input device using a translational scaling factor and without using the rotational scaling factor; and
generate the control signal in dependence on the scaled translational movement of the surgeon input device.

There may be provided a surgical robotic system comprising:
a surgical robot arm comprising a series of joints by which its configuration can be altered, the surgical robot arm having an attachment for a surgical instrument at a distal end of the surgical robot arm;
a surgeon input device; and
a control system as described herein.

There is provided a method of controlling a surgical robot arm in a surgical robotic system, the surgical robotic system comprising the surgical robot arm and a surgeon input device, wherein the surgical robot arm comprises: (i) a series of joints by which its configuration can be altered, and (ii) an attachment for a surgical instrument at a distal end of the surgical robot arm, the method comprising:
identifying a condition that suggests that the surgical robot arm is being used for a surgical step involving rotation of a surgical instrument attached to the surgical robot arm;
in response to identifying said condition, outputting a prompt for prompting a user to enable a rotational scaling mode;
receiving a mode selection input that indicates that the user has selected to enable the rotational scaling mode;
enabling the rotational scaling mode in response to receiving the mode selection input; and
when the rotational scaling mode is enabled:
   receiving a control input from the surgeon input device;
   using the received control input to determine an indication of a movement of the surgeon input device, wherein the indication of the movement comprises a matrix representation of a rotation of the surgeon input device;
   converting the matrix representation into an axis-angle representation of the rotation of the surgeon input device, wherein said axis-angle representation of the rotation comprises: (i) an indication of a rotation axis for the rotation, and (ii) an angle of rotation about the rotation axis;
   scaling the angle of rotation with a rotational scaling factor to determine a scaled angle of rotation about the rotation axis for a scaled axis-angle representation of a scaled rotation;
   generating a control signal for the surgical robot arm using the scaled axis-angle representation of the scaled rotation; and
   causing the generated control signal to be sent to the surgical robot arm in order to drive the surgical robot arm such that its configuration is altered.

There is provided a computer readable storage medium having stored thereon computer readable instructions that, when executed at a control system for a surgical robotic system, cause the control system to perform any of the methods described herein.

The above features may be combined as appropriate, as would be apparent to a skilled person, and may be combined with any of the aspects of the examples described herein.

### BRIEF DESCRIPTION OF THE DRAWINGS

Examples will now be described in detail with reference to the accompanying drawings in which:

FIG. 1 shows an example surgical robotic system;

FIG. 2 shows an example surgical instrument;

FIG. 3 shows an example surgical robot arm;

FIG. 4 shows an example surgical robotic system;

FIG. 5 is a flow chart for a surgical robot arm calibration process;

FIGS. 6a and 6b show a flow chart for a method of controlling a surgical robot arm using a control system;

FIG. 7 shows an example surgeon input device comprising a hand controller connected to a gimbal assembly; and

FIG. 8 illustrates an axis-angle representation of a rotation.

The accompanying drawings illustrate various examples. The skilled person will appreciate that the illustrated element boundaries (e.g., boxes, groups of boxes, or other shapes) in the drawings represent one example of the boundaries. It may be that in some examples, one element may be designed as multiple elements or that multiple elements may be designed as one element. Common reference numerals are used throughout the figures, where appropriate, to indicate similar features.

### DETAILED DESCRIPTION

The following description is presented by way of example to enable a person skilled in the art to make and use the invention. The present invention is not limited to the embodiments described herein and various modifications to the disclosed embodiments will be apparent to those skilled in the art.

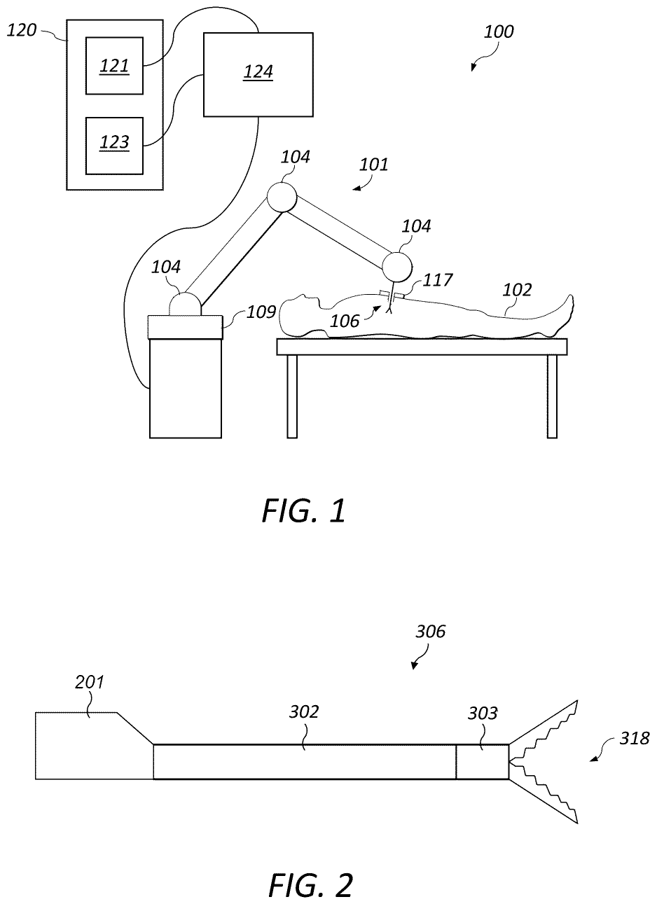

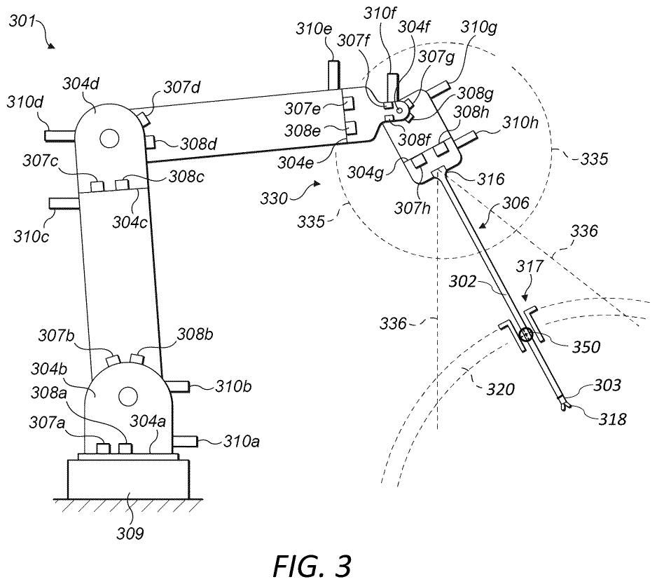

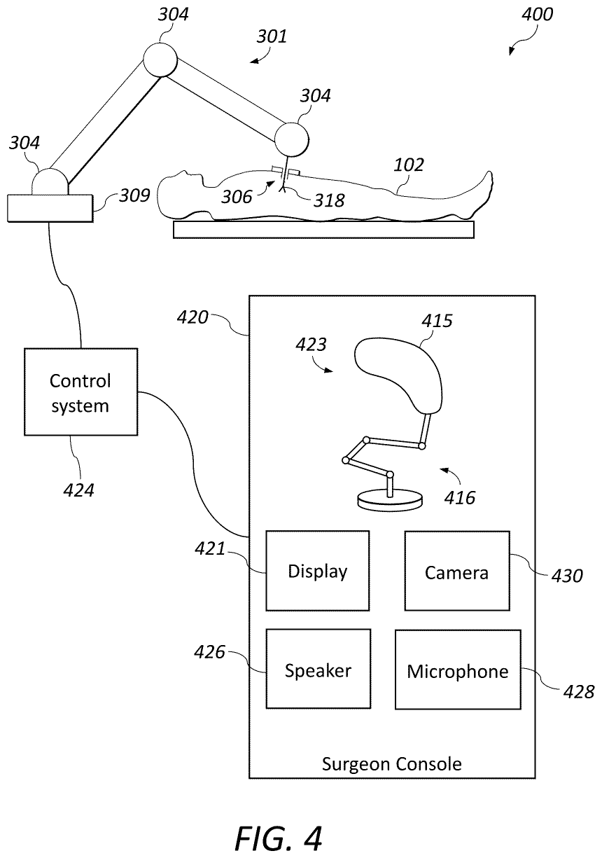

FIG. 3 shows an example of a surgical robot arm 301 which can operate within a surgical robotic system—such as the surgical robotic system shown in FIG. 1, or the surgical robotic system shown in FIG. 4 as will be described in further detail herein.

At its proximal end, the surgical robot arm 301 comprises a base 309. The surgical robot arm has a series of rigid arm members. Each arm member in the series is joined to the preceding arm member by a respective joint 304—shown in FIG. 3 as joints 304a-g. Joints 304a-g may be referred to as a series of joints. Joints 304a-e and 304g are revolute joints. Joint 304f is composed of two revolute joints whose axes are orthogonal to each other, e.g. as in a “Hooke's” or universal joint. Joint 304f may be termed a “wrist joint”. A surgical robot arm could be jointed differently from the surgical robot arm 301 of FIG. 3. For example, joint 304d could be omitted and/or joint 304f could permit rotation about a single axis. Alternatively, or additionally, the surgical robot arm 301 could include one or more joints that permit motion other than rotation between respective sides of the joint, such as a prismatic joint by which an instrument attachment can slide linearly with respect to more proximal parts of the surgical robot arm.

The joints are configured such that the configuration of the surgical robot arm can be altered. This allows the distal end 330 of the surgical robot arm to be moved to an arbitrary point in a three-dimensional working volume illustrated generally at 335. One way to achieve that is for the joints to have the arrangement illustrated in FIG. 3. Other combinations and configurations of joints could achieve a similar range of motion. There could be more or fewer arm members.

The distal end 330 of the surgical robot arm 301 has an attachment 316 by means of which a surgical instrument 306 can be releasably attached. Movement of the surgical robot arm 301 thereby causes movement of the surgical instrument 306. The surgical instrument 306 has a shaft 302. The surgical instrument 306 has an end effector 318 at the distal end of the shaft 302. The end effector 318 consists of a device for engaging in a procedure, for example a cutting, grasping, holding or imaging device. As described herein, terminal joint 304g may be a revolute joint. The surgical instrument 306 and/or the attachment 316 may be configured so that the surgical instrument 306 (e.g. in particular, its shaft 302) extends linearly parallel with the rotation axis of the terminal joint 304g of the surgical robot arm 301. In this example the surgical instrument 306 (e.g. in particular, its shaft 302) extends along an axis coincident with the rotation axis of joint 304g.

For some types of minimally invasive surgical procedure, the surgical instrument 306 may access a surgical site within the patient's body through a synthetic access port 317. For example, the minimally invasive procedure may be performed at a surgical site within the patient's abdomen. The port 317 may provide a passageway through the outer tissues 320 of the patient so as to limit disruption to those tissues as the surgical instrument 306 is inserted and removed, and as the surgical instrument 306 is moved by the surgical robot arm 301 about the surgical site. For other types of minimally invasive surgical procedure, the surgical instrument 306 may access a surgical site within the patient's body directly through a natural orifice. For example, the minimally invasive procedure may be performed at a surgical site in the patient's throat, and the natural orifice may be the patient's mouth.

FIG. 2 shows in more detail an example surgical instrument for attachment to the surgical robot arm 301 shown in FIG. 3. The surgical instrument 306 comprises a base 201 at its proximal end by which it connects to (e.g. attaches to) the surgical robot arm 301. A shaft 302 connects the base 201 to an articulation 303. The shaft 302 is a rigid linear shaft. The articulation 303 is connected to the distal end of the shaft 302. The articulation 303 connects the shaft 302 to an end effector 318. The end effector 318 is at the distal end of the surgical instrument 306. By way of example only, in FIG. 2, a pair of serrated jaws are illustrated as the end effector 318. As another example, the instrument 306 may be a needle holder and the end effector 318 may have a pair of jaws with textured opposing surfaces, e.g. for gripping a suturing needle. The articulation 303 permits the end effector 318 to move relative to the shaft 302. The skilled person would be aware of numerous articulations suitable for permitting the end effector 318 to move relative to the shaft 302, and so for conciseness the specific implementation of the articulation will not be discussed further herein.

It is to be understood that FIG. 2 shows just one specific example of a surgical instrument, and that various other suitable surgical instruments exist to which the principles described herein could be applied.

Returning to FIG. 3, joints 304e and 304f of the surgical robot arm 301 are configured so that, with the distal end 330 of the surgical robot arm 301 held at an arbitrary location in the working volume 335, the surgical instrument 306 can be moved in an arbitrary direction within a cone 336. One way to achieve that is for the terminal part of the arm to comprise the pair of joints 304e and 304f whose axes are mutually arranged as described above. Other mechanisms can achieve a similar result. For example, joint 304g could influence the attitude of the instrument if the instrument extends in a direction which is not parallel to the axis of joint 304g.

The surgical robot arm 301 comprises a series of motors 310a-h. With the exception of the compound joint 304f, which is served by two motors, each motor is arranged to drive rotation about a respective joint of the surgical robot arm 301. The motors are controlled by a control system (such as control system 124 shown in FIG. 1, or the control system 424 shown in FIG. 4 as will be described in further detail herein). The control system comprises a processor and a memory. The memory stores, in a non-transient way, software code that can be executed by the processor to cause the processor to control the motors 310a-h in order to alter the configuration of the surgical robot arm 301 in the manner described herein.

The surgical robot arm 301 may comprise a series of sensors 307a-h and 308a-h. These sensors may comprise, for each joint, a position sensor 307a-h for sensing the rotational position of the joint and a force sensor 308a-h for sensing forces (or torques) applied about the joint's rotation axis. Compound joint 304f may have two pairs of sensors. One or both of the position and force sensors for a joint may be integrated with the motor for that joint. The outputs of the sensors are passed to the control system (such as control system 124 shown in FIG. 1, or the control system 424 shown in FIG. 4 as will be described in further detail herein) where they form inputs for the processor.

It is to be understood that FIG. 3 shows just one specific example of a surgical robot arm, and that various other suitable surgical robot arms exist to which the principles described herein could be applied.

FIG. 4 shows an example a surgical robotic system 400 comprising a surgical robot arm 301, a control system 424 and a surgeon console 420. FIG. 4 shows a surgical instrument 306 attached to the distal end of the surgical robot arm 301 during a surgical procedure carried out on a patient 102, as described above. The surgeon console 420 comprises a surgeon input device 423 and a display 421. A video feed of the surgical site may be captured by an endoscope, often attached to a further surgical robot arm (not shown in FIG. 4 for simplicity), and displayed at the display 421 of the remote surgeon console. As shown in FIG. 4, the surgeon console 420 may also comprise other output devices (e.g. a speaker 426) for conveying information to the surgeon and/or other input devices (e.g. a microphone 428) for receiving inputs from the surgeon. The surgeon console 420 may also comprise a camera 430 for capturing one or more images (e.g. a video stream) of the surgeon.

A simplified schematic of the surgical robot arm 301 is shown in FIG. 4 for ease of illustration. It is to be understood that the surgical robot arm 301 shown in FIG. 4 can have the same properties and features as the surgical robot arm 301 described with reference to FIG. 3.

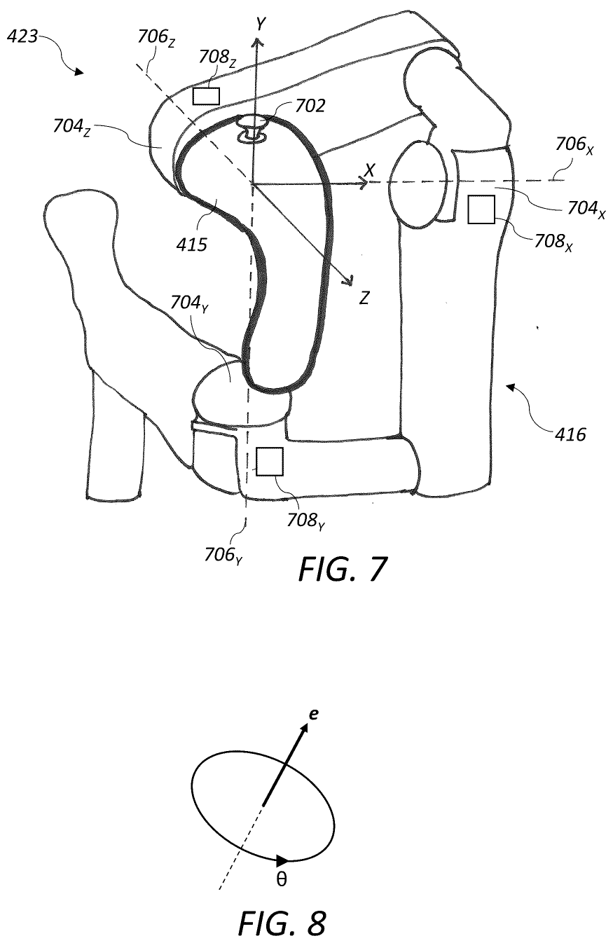

In FIG. 4, the surgeon input device 423 comprises a hand controller 415 connected to a gimbal assembly 416 which permits the hand controller 415 to move, e.g. with six degrees of freedom such that the hand controller 415 can change position by translation in three perpendicular axes of the reference frame of the hand controller (e.g. backward/forward, up/down and left/right) and such that the hand controller 415 can change orientation through rotation about three perpendicular axes (e.g. yaw, pitch and roll). A simplified schematic of the gimbal assembly is shown in FIG. 4 for ease of illustration, whilst a more detailed illustration of an example of the gimbal assembly is shown in FIG. 7 and is described in more detail below. The configuration of the gimbal assembly 416 can be detected by sensors on the gimbal assembly 416 (e.g. by measuring the positions of the joints of the gimbal assembly) and an indication of the gimbal assembly configuration can be passed to the control system 424. A surgeon can move the hand controller 415 in order to request corresponding movement of the surgical instrument 306 attached to the surgical robot arm 301. The skilled person would be aware of numerous gimbal assemblies suitable for permitting the hand controller 415 to move with six degrees of freedom and for detecting that movement (one example of which is shown in FIG. 7). In an alternative example, instead of the gimbal assembly 416, the hand controller 415 could be equipped with accelerometers which permit its position and orientation to be estimated. In the example shown in FIG. 4 the surgeon console 420 comprises just one surgeon input device 423, but this is just for simplicity of illustration, and in other examples the surgeon console 420 may comprise two or more surgeon input devices, which each have corresponding features to those of surgeon input device 423 described herein. For example, the surgeon console 420 may comprise two surgeon input devices, each of which comprises a hand controller connected to a gimbal assembly so that a surgeon can operate each of the two surgeon input devices using one of their hands, e.g. for controlling two surgical robot arms in the surgical robotic system.

A control system 424 connects the surgeon input device 423 to the surgical robot arm 301. The control system 424 may be separate from the remote surgeon console 420 and the surgical robot arm 301. The control system 424 may be collocated with the remote surgeon console. The control system 424 may be collocated with the surgical robot arm 301. The control system 424 may be distributed between the remote surgeon console and the surgical robot arm 301.

The control system 424 comprises a processor and a memory. The memory stores, in a non-transient way, software code that can be executed by the processor to cause the processor to control the surgical robot arm 301 in the manner described herein.

The control system 424 receives inputs from the surgeon input device 423 and converts those inputs to control signals to move one or more of the joints 304 of the surgical robot arm 301 in order to alter its configuration. The control signals can be generated by implementing inverse kinematics. In other words, the control signals can be generated based on kinematic equations that define the relationship between the position of the one or more joints 304 and the position of the attachment 316 for the surgical instrument-as would be well understood by the skilled person. The control system 424 sends these control signals to the surgical robot arm 301, where the corresponding one or more of the joints 304 are driven accordingly. Movement of the surgical instrument 306 attached to the surgical robot arm 301 can thereby be controlled by the control system 424 in response to movement of the surgeon input device 423.

The control system 424 can use a control relationship to transform an input from the surgeon input device 423 so as to generate a control signal for causing movement of the surgical instrument 306. In accordance with the control relationship, the position and orientation of a surgeon input device 423 dictates the position and orientation of the surgical instrument 306 (e.g. in particular, a part of the surgical instrument 306 such as the distal end of its shaft 302 or its end effector 318). For example, referring to FIG. 4, the position and orientation of the hand controller 415 in the hand controller workspace can be directly converted to a position and orientation of the end effector 318 in the end effector workspace. More specifically, the control system 424 may determine the position and orientation of the hand controller 415 at a plurality of instances in time. The position and orientation of the hand controller 415 can be determined (e.g. by implementing forward kinematics) using the inputs from the sensors on the gimbal assembly 416. The position and orientation of the hand controller 415 can be determined irregularly or at predetermined time intervals, e.g. every 0.4 milliseconds (ms). In other words, the position and orientation of the hand controller 415 can be determined at a predetermined frequency, e.g. of 2.5 kHz. The difference in the position and/or orientation of the hand controller between two instances in time can be determined. In response, using a control relationship, the control system 424 may generate a control signal in order to drive the surgical robot arm 301 such that its configuration is altered so as to cause a corresponding change to the position and/or orientation of the end effector of the surgical instrument.

Constraints may be placed on the movement of the surgical instrument that can be caused by the control system. One such constraint is that the control system 424 is configured to control the surgical robot arm 301, in dependence on inputs received at the surgeon input device 423, to alter the configuration of the surgical robot arm 301 whilst maintaining an intersection between the surgical instrument 306 attached to the surgical robot arm 301 and a pivot point. The control system may be configured to control the surgical robot arm 301 in this way during a minimally invasive procedure. FIG. 3 shows an example pivot point 350. The pivot point 350 is a point in space about which the control system 424 is configured to cause the surgical instrument 306 to pivot. The pivot point may be referred to as a “virtual pivot point” or a “fulcrum”. The pivot point 350 may be mechanically enforced or may be a software constraint enforced by the control system 424. In the example shown in FIG. 3, there is nothing physically present at the pivot point (which is why it may be referred to as a virtual pivot point or a virtual fulcrum) and the pivot point 350 is a software constraint enforced by the control system 424 when it determines the control signals for driving the surgical robot arm 301.

As such, the surgical robot arm 301 is driven using control signals which maintain an intersection between the surgical instrument 306 and the pivot point 350. For example, during the minimally invasive procedure, the surgeon can use the surgeon input device 423 to indicate a desired position and orientation of the end effector 318 of the surgical instrument 306. In response, the control system 424 determines a configuration of the series of joints 304 of the surgical robot arm 301 that will result in both (i) the end effector 318 of the surgical instrument 306 being placed in that desired position and orientation and (ii) the shaft 302 of the surgical instrument 306 passing through (e.g. maintaining an intersection with) the pivot point 350, and to generate a control signal to move the series of joints 304 to that configuration.

By determining a suitable pivot point, the disruption to the outer tissues 320 of the patient caused by moving the surgical instrument 306 during a minimally invasive procedure can be minimised. For example, a suitable pivot point 350 may be located within the port 317, e.g. at or close to the centre of the port 317.

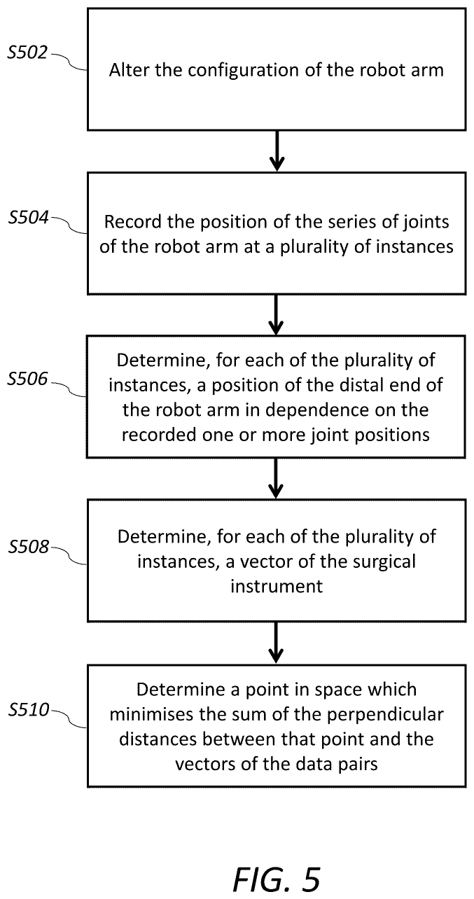

As an example, a calibration process can be performed prior to performing a minimally invasive procedure in order to determine a suitable pivot point. FIG. 5 shows an example surgical robot arm calibration process. The operating mode of the surgical robotic system 400 can be set to be a calibration mode during the calibration process so that the surgical robotic system can act accordingly in order to calibrate the surgical robot.

In step S502, the configuration of the surgical robot arm 301 can be altered whilst the surgical instrument 306 is inside the access port 317 or natural orifice. During the calibration process, the configuration of the surgical robot arm 301 can be altered by the application of external forces directly onto the surgical robot arm 301. For example, the user (e.g. a surgeon or a member of the operating room staff) may apply forces directly to the surgical robot arm 301 (e.g. by pushing a joint of the surgical robot arm 301)—which can be sensed by the force sensors 308a-h and acted on by the control system 424 in a manner that would be understood by the skilled person. During the calibration process, when operating in the calibration mode, the control system 424 can control the surgical robot arm 301 to maintain a position in which it is placed by means of external forces applied directly to the surgical robot arm 301.

During the calibration process, when operating in the calibration mode, the surgical robot arm 301 can be moved generally transversely to the shaft 302 of the surgical instrument 306. The configuration of the surgical robot arm 301 may be altered such that the distal end of the surgical robot arm 301 is moved in two dimensions transverse (e.g. perpendicular) to the shaft 302: e.g. with (i) components parallel to a direction that is transverse (e.g. perpendicular) to the shaft 302 and also with (ii) components orthogonal to that direction but transverse (e.g. perpendicular) to the shaft 302. To do this, the user (e.g. a surgeon or a member of the operating room staff) may gyrate the distal end 330 of the surgical robot arm 301 about a point generally aligned with the natural axis of the access port or natural orifice. This causes the surgical instrument 306 to come into contact with the access port 317 (or natural orifice) such that the access port 317 (or natural orifice) applies a lateral force on the shaft 302. That force can be accommodated by motion about the joint 304f. The force is “lateral” in the sense that it is applied to the sides of the instrument and is generally in a direction that is transverse (e.g. perpendicular) to the shaft 302 of the instrument 306.

In step S504, as the configuration of the surgical robot arm 301 is being altered, the position sensors 307a-h can record the position of each joint 304 of the series of joints of the surgical robot arm 301. The position sensors 307a-h can record the positions of each joint of the surgical robot arm 301 at a plurality of instances in time. Position information may be recorded irregularly or at predetermined time intervals, e.g. every 20 milliseconds (ms). In other words, position information may be recorded at a predetermined frequency, e.g. of 50 Hz. The position sensors provide the recorded position information to the control system 424. The control system may also store in memory information indicating one or more parameters of the surgical instrument 306 (e.g. including the length of the shaft and/or the orientation of its shaft relative to its base). These parameters of the surgical instrument 306 may be read from a memory on the surgical instrument itself and passed to the control system 424.

In steps S506 and S508, the control system 424 uses this information to determine, at each of the plurality of instances in time: (a) the position of the distal end 330 of the surgical robot arm 301 relative to the base 309 and (b) a vector representing the surgical instrument 306 (e.g. in particular, its shaft 302) relative to the distal end 330 of the surgical robot arm 301. Position (a) and vector (b) may be termed a data pair.

The vectors of the data pairs will approximately (but usually not exactly) converge, from their respective distal end position, on the natural rotation centre of the access port 317 or natural orifice. By collecting a plurality of said data pairs, and then solving for a best estimate (i.e. an estimate with the least error) of a location where the vectors converge, the control system 424 can determine a fulcrum (e.g. a pivot point) within the access port or natural orifice. For example, in step S510, the control system 424 may estimate, as the pivot point, the point in space which minimises the sum of the perpendicular distances between that point and the vectors of the data pairs. Here, a “perpendicular distance” between the point and a vector refers to the distance between the point and the vector in a direction perpendicular to the vector. Therefore, the perpendicular distance between the point and a vector is the distance between the point and the position on the vector which is closest to the point. In some examples, the control system 424 may estimate, as the pivot point, the point in space which minimises the sum of the squares of the perpendicular distances between that point and the vectors of the data pairs. The control system 424 can store this pivot point in memory for later use.

In the examples described above, the relationship between the movement of the hand controller 415 and the resultant movement of the end effector 318 is 1:1, i.e. when the surgeon moves the hand controller 415 in a certain way the control system 424 will drive the surgical robot arm 301 such that the end effector 318 moves in the same way as the hand controller 415. However, when using the surgeon input device 423, motion of the hand controller 415 may be restricted. For example, the end effector 318 may have a larger range of motion than the surgeon input device 423 and/or than the wrist of the surgeon operating the surgeon input device 423. The translational motion of the hand controller 415 may be restricted, e.g. displacement of the hand controller 415 may be limited by the arrangement of the gimbal assembly 416. Furthermore, the rotational motion of the hand controller 415 may be limited. In particular, the human wrist (of the surgeon) cannot rotate infinitely and the physical parts of the system (e.g. the surgeon, the hand controllers and the gimbal assemblies) cannot pass through each other.

Some surgical steps (e.g. suturing) involve performing a single rotational movement of the end effector that is greater than the rotational range of the hand controller 415 of the surgeon input device 423 (e.g. due to the physical limitations of the surgeon and/or the physical parts of the surgeon input device 423). In previous systems, in order to perform these rotational movements, the surgeon may rotate the hand controller 415 as much as they can before reaching a limit and then use a clutch to decouple the hand controller 415 from the surgical robot arm 301 that it is controlling so that the hand controller 415 can be moved to a different position/orientation away from the limit that has been reached without moving the surgical robot arm 301. The surgeon can then release the clutch to recouple the hand controller 415 to the surgical robot arm 301 so that the remaining part of the rotational movement can be performed. As an example, the rotational movement involved for suturing is approximately 270° at the end effector, but the hand/hand controller is only capable of rotating by up to approximately 180° without having to use the clutch. Surgeons find that having to clutch and reorient their hand during a compound rotation motion feels unnatural, adds delay into the process, and can sometimes make the rotation harder to control.

As such, in examples described below, a rotational scaling factor is applied so that rotations of the hand controller 415 can be mapped to larger rotations of the end effector 318. For example, in order to control the surgical robot arm 301, changes in the joint positions of the gimbal assembly 416 are measured and used to calculate a change in the pose of the hand controller 415 (e.g. using forward kinematics, as is known in the art). This change in the pose of the hand controller 415 is mapped to a change in pose of the surgical robot arm 301 whose desired joint positions (for implementing the change in pose) are calculated (e.g. using inverse kinematics, as is known in the art). Translational changes in the pose of the hand controller 415 can be scaled by simply multiplying them with a translational scaling factor. However, scaling up rotational changes in the pose of the hand controller 415 is not so simple. For example, an indication of a movement of the surgeon input device 423 comprises a matrix representation of a rotation of the surgeon input device 423. The matrix representation of the rotation has components for the axes (denoted X, Y and Z in FIG. 7) of the reference frame for the surgeon input device 423. Simply scaling the components in the matrix representation of the rotation would not produce a correctly scaled rotation. Instead, in examples described herein the matrix representation of the rotation of the surgeon input device 423 is converted into an axis-angle representation of the rotation of the surgeon input device 423, which can then be scaled.

As illustrated in FIG. 8, an axis-angle representation of a rotation parameterizes a rotation in a 3D space (e.g. in the reference frame of the surgeon input device 423) by two quantities: a unit vector e indicating the direction of a single axis of rotation in the 3D space, and an angle θ describing the magnitude of the rotation about the axis. The angle θ can be multiplied with a rotational scaling factor to determine a scaled angle of rotation about the axis. A scaled axis-angle representation of the scaled rotation comprises: (i) an indication of the rotation axis for the rotation and (ii) the scaled angle of rotation about the rotation axis. This scaled axis-angle representation of the scaled rotation can then be converted back to a matrix representation and used for controlling the surgical robot arm 301 as described below.

In examples described herein a rotational scaling mode (in which the angle of rotation is scaled with a rotational scaling factor) can be enabled and disabled by a user (e.g. the surgeon). It is the user's decision to enable or disable the rotational scaling mode. This is in contrast to automatically enabling or disabling the rotational scaling mode, which could be detrimental to patient safety, e.g. if the surgeon is not expecting the change of mode, and/or which could be unwanted by the surgeon. Although it is ultimately the user's (e.g. the surgeon's) choice whether to enable or disable the rotational scaling mode, in examples described below, the control system 424 can prompt the user to enable or disable the rotational scaling mode in response to identifying a condition that suggests that the surgical robot arm 301 is being used for a surgical step involving rotation (e.g. suturing). Issuing a prompt in situations in which a change of mode is likely to be helpful, facilitates the change of mode to the rotational scaling mode, thereby reducing the disruption caused by the change of mode to the workflow of a surgical procedure.

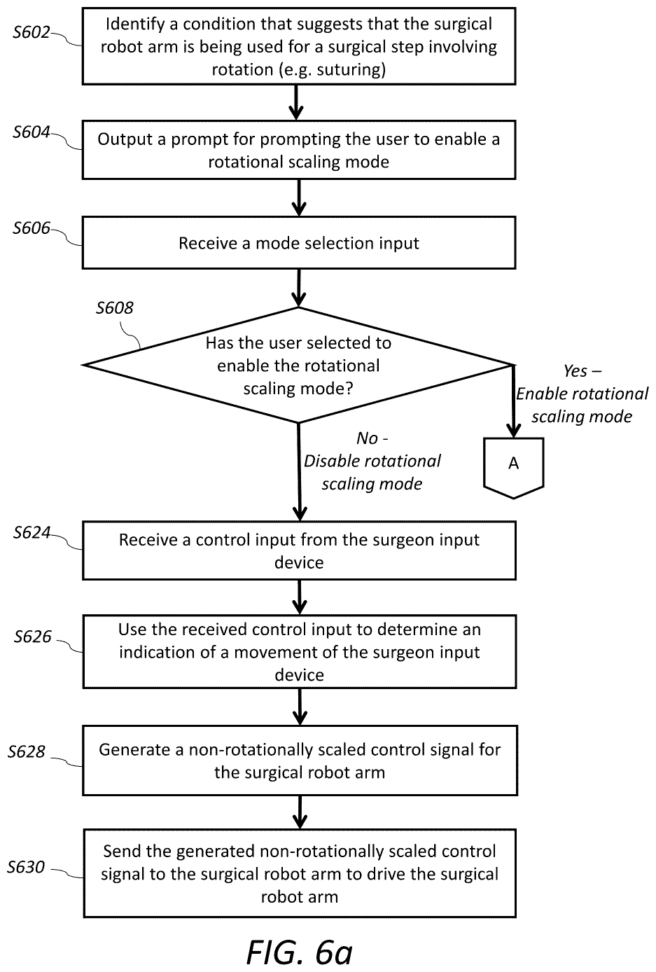

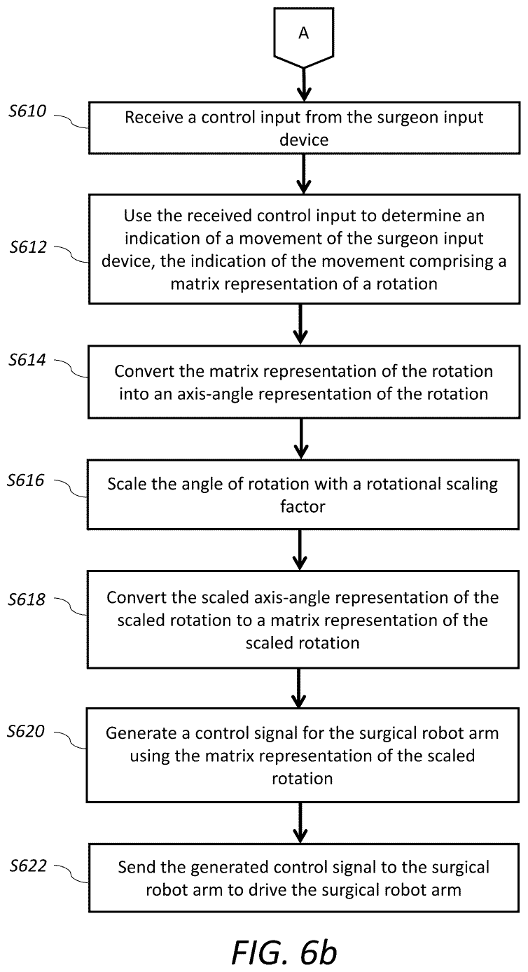

FIGS. 6a and 6b show a flow chart for a method of controlling the surgical robot arm 301 in the surgical robotic system 400. The control system 424 of the surgical robotic system 400 shown in FIG. 4 can be configured to perform this method.

In step S602 the control system 424 identifies a condition that suggests that the surgical robot arm 301 is being used for a surgical step (e.g. suturing) involving rotation of the surgical instrument 306. A needle holder is a type of surgical instrument which is used for gripping (i.e. holding) objects such as suturing needles (which are curved needles). During a step of suturing, one or more needle holders are used to hold a suturing needle, and a needle holder is rotated through approximately 270 degrees to pass the suturing needle through some tissue, thereby pulling some suturing thread through the tissue.

As a first example, in step S602, the control system 424 may identify a condition that suggests that the surgical robot arm 301 is being used for suturing by determining that the surgical robotic system 400 currently has one or more (e.g. two) operational surgical robot arms 301 with needle holder instruments attached thereto. Since needle holders are used for suturing, the attachment of one or more needle holders to surgical robot arms which are operational (e.g. coupled to, and being controlled by, the surgeon input device(s) in the surgical robotic system 400) may suggest that the surgical robot arm 301 is being used for suturing.

As a second example, in step S602, the control system 424 may identify a condition that suggests that the surgical robot arm 301 is being used for suturing by determining that the surgical robotic system 400 currently has two or more operational surgical robot arms with a predetermined combination of surgical instruments attached thereto, where the predetermined combination of surgical instruments is suitable for performing suturing. Some combinations of surgical instruments (e.g. a needle holder attached to one surgical robot arm and a grasper attached to another surgical robot arm) may be suitable for performing suturing, so the attachment of one of these predetermined combinations of surgical instruments to surgical robot arms which are operational may suggest that the surgical robot arm 301 is being used for suturing.

As a third example, in step S602, the control system 424 may identify a condition that suggests that the surgical robot arm 301 is being used for suturing by determining that the current time in a surgical procedure matches a time at which suturing is planned to be performed in the surgical procedure. Surgical procedures are planned very accurately and precisely, so for some surgical procedures (e.g. well known, routine procedures) the time within the surgical procedure at which suturing is performed can be predicted accurately. For example, suturing will often occur near the end of the surgical procedure when previously made incisions are being stitched back up.

As mentioned above, a video feed of the surgical site may be captured by an endoscope in the surgical robotic system. As a fourth example, in step S602, the control system 424 may identify a condition that suggests that the surgical robot arm 301 is being used for suturing by analysing one or more images of the surgical site captured by the endoscope. For example, a machine learning algorithm could be trained to identify when suturing is being performed based on a set of training images (or video sequences) of surgical sites in which suturing is occurring. For example, the algorithm may pick up on certain features in the images (e.g. the presence of a suturing needle, suturing thread, a needle holder, an incision in tissue to be stitched up, etc.) in order to predict that suturing is being performed.

As a fifth example, in step S602, the control system 424 may identify a condition that suggests that the surgical robot arm 301 is being used for suturing by determining that a speed and/or pattern of motion of the surgical robot arm 301 matches that expected during suturing. The speed and/or pattern of motion of the surgical robot arm 301 may be determined based on one or more of: (i) the control signals sent to the surgical robot arm 301 for controlling its motion, (ii) signals received from the position sensors 307 and/or torque sensors 308 on the surgical robot arm 301, and (iii) one or more images of the surgical site captured by the endoscope.

In response to identifying the condition in step S602, in step S604 the control system 424 outputs a prompt for prompting the user (e.g. the surgeon) to enable a rotational scaling mode. For example, in step S604 the control system 424 could cause a visual indication to be displayed (e.g. on the display 421 at the surgeon console 420) prompting the user to enable the rotational scaling mode. For example, the visual indication could be a message saying “Would you like to enable rotational scaling?” and giving the user an option to select ‘yes’ or ‘no’. As another example, the visual indication could be a symbol or icon which may be displayed to indicate to the surgeon that they might want to consider enabling the rotational scaling mode.

As another example, in step S604 the control system 424 could cause an audio indication to be output (e.g. from the speaker 426 of the surgeon console 420) prompting the user to enable the rotational scaling mode. For example, the audio indication could be an audio message saying “Would you like to enable rotational scaling?”. As another example, the audio indication could be a distinctive tone or sequence of tones that the user can be trained to interpret as a prompt to enable the rotational scaling mode.

In step S606 the control system 424 receives a mode selection input that indicates whether the user (e.g. the surgeon) has selected to enable the rotational scaling mode. In particular, the control system 424 receives the mode selection input responsive to the user making a selection on the surgeon console 420 to enable the rotational scaling mode. The user may make the selection in different ways in different examples. For example, the user may select an option displayed on the display 421 of the surgeon console 420. If the display 421 is a touch screen then the user may select the option by touching it on the display 421. The user could alternatively select the option using a mouse or keyboard to navigate to it on the display 421 and then select it. The option may be accessed via a menu displayed on the display 421.

In some examples, in step S606 the surgeon can make a selection via the surgeon console 420 to enable the rotational scaling mode whilst keeping each of their hands on a hand controller. This means that making the selection causes minimal disruption or interruption to the surgical procedure. For example, in step S606 the user (e.g. the surgeon) may issue a voice command which is received by the microphone 428 of the surgeon console 420. To give some examples, the user may say “Enable the rotational scaling mode” or may answer “Yes” in response to an audio indication output in step S604 saying “Would you like to enable rotational scaling?”. The surgeon console 420 may include a processing unit for analysing and interpreting the voice command received from the user. The processing unit might only analyse audio signals received by the microphone 428 for the purposes of trying to interpret a voice command within a time window (e.g. 5 seconds) following the output of the prompt in step S604.

As another example of how the surgeon can make the selection to enable the rotational scaling mode whilst keeping both of their hands on the hand controllers, in step S606 the user (e.g. the surgeon) may perform a gesture (e.g. a head motion, such as a nod) which is identified in images of the user captured by the camera 430 of the surgeon console 420. The surgeon console 420 may include a processing unit for analysing and interpreting images of the surgeon captured by the camera 430 in order to identify the gesture. The processing unit might only analyse images captured by the camera 430 for the purposes of trying to identify the gesture within a time window (e.g. 5 seconds) following the output of the prompt in step S604.

As well as enabling or disabling the rotational scaling mode, the user may select a rotational scaling factor (e.g. from a set of predetermined rotational scaling factors, e.g. 1, 1.5 or 2). As such, the mode selection input may indicate the rotational scaling factor based on a user selection of the rotational scaling factor. It is noted that a rotational scaling factor of 1 corresponds to there being no scaling of the rotation. In examples described herein, the rotational scaling factor is positive so that the direction of a rotation is not altered by applying the rotational scaling factor (i.e. only the magnitude of the rotation is altered by applying the rotational scaling factor). For example, the rotational scaling factor may be greater than 1 (e.g. it may be between 1.1 and 3) for scaling up the rotation, i.e. for increasing the magnitude of the rotation of the end effector 318. To give two examples, the rotational scaling factor may be 1.5 or 2. The rotational scaling factor may be less than 1 (e.g. it may be between 0.3 and 0.9) for scaling down the rotation, i.e. for decreasing the magnitude of the rotation of the end effector 318. To give an example, the rotational scaling factor may be 0.5. Scaling down the rotation may be useful for increasing the precision with which the surgeon can control the end effector 318.

In step S608 the control system 424 determines, based on the mode selection input received in step S606, whether the user has selected to enable the rotational scaling mode. In response to receiving a mode selection input that indicates that the user has selected to enable the rotational scaling mode the control system 424 enables the rotational scaling mode, and the method passes to step S610 (shown in FIG. 6b). In response to receiving a mode selection input that indicates that the user has selected to disable the rotational scaling mode the control system 424 disables the rotational scaling mode (which may be considered to be equivalent to setting the rotational scaling factor to 1), and the method passes to step S624.

Steps S610 to S622, shown in FIG. 6b, are performed when the rotational scaling mode is enabled. In step S610 the control system 424 receives a control input from the surgeon input device 423. The control input indicates a requested movement of the surgical instrument 306. As described herein with reference to FIG. 4, the surgeon input device 423 may comprise a hand controller 415 that the surgeon can move in order to request corresponding movement of the surgical instrument 306 attached to the surgical robot arm 301. FIG. 7 shows more detail of an example surgeon input device 423 comprising a hand controller 415 connected to a gimbal assembly 416.

The hand controller 415 is arranged to fit comfortably in the palm of the surgeon's hand, and has a thumbstick 702 on the top which the surgeon can use with their thumb whilst holding the hand controller 415 with the rest of their hand. The hand controller 415 may also comprise a trigger (e.g. for controlling jaws of a surgical instrument) which is arranged so that the surgeon can comfortably actuate the trigger with one or more of their fingers whilst holding the hand controller 415. The hand controller 415 may also comprise one or more buttons, e.g. a clutch button for engaging or disengaging the clutch and/or an electrosurgery button for activating or deactivating an electrosurgical instrument. The gimbal assembly 416 comprises a plurality of rigid arm segments connected by joints 704x, 704Y and 704z. Each of the joints allows the hand controller 415 to rotate about an axis, and movement about two or more of the axes at the same time allows for translational movement of the hand controller 415. The axes of a reference frame of the surgeon input device 423 are shown as X, Y and Z in FIG. 7, and it can be seen that rotation about the joint 704x will rotate the hand controller 415 about the X axis (i.e. about the dashed line 706x which passes through the joint 704x), rotation about the joint 704Y will rotate the hand controller 415 about the Y axis (i.e. about the dashed line 706Y which passes through the joint 704y) and rotation about the joint 704z will rotate the hand controller 415 about the Z axis (i.e. about the dashed line 706z which passes through the joint 704z). The surgeon input device 423 shown in FIG. 7 is arranged so that the origin of the coordinate system (i.e. where X=Y=Z=0) is at a position within the hand controller 415, and will always be at a position with the hand controller 415 as the surgeon moves the hand controller 415. This helps to create a natural feel to the way in which the hand controller 415 moves. The gimbal assembly 416 also comprises position sensors (708x, 708Y and 708z) for the respective joints (704x, 704Y and 704z) which can measure the position of the joints. The control input received in step S610 comprises indications of joint positions of the gimbal assembly, as measured by the position sensors 708x, 708Y and 708z.

In step S612 the control system 424 uses the received control input to determine an indication of a movement of the surgeon input device 423. The indication of the movement comprises a matrix representation of a rotation of the surgeon input device 423. In particular, the matrix representation of the rotation of the surgeon input device 423 represents the rotation in the reference frame of the surgeon input device 423, thereby representing a three dimensional rotation in the coordinate system of the 3D reference frame of the surgeon input device 423. The matrix representation of the rotation of the surgeon input device 423 has components in the X, Y and Z dimensions. The control system may determine the indication of a movement of the surgeon input device 423 by implementing forward kinematics. A person skilled in the art would be aware of how to implement forward kinematics, and for conciseness the details of how to implement forward kinematics are not included herein.

Using the control input received from the surgeon input device 423, the control system 424 may determine the position and orientation of the hand controller 415 at a plurality of instances in time using the inputs from the position sensors 708x, 708Y and 708z. The position and orientation of the hand controller 415 can be determined irregularly or at predetermined time intervals, e.g. every 0.4 milliseconds (ms). In other words, the position and orientation of the hand controller 415 can be determined at a predetermined frequency, e.g. of 2.5 kHz. The difference in the position and/or orientation of the hand controller between two instances in time can be determined. For example, it may be determined that in the preceding 0.4 ms interval the orientation of the hand controller 415 changed by 1 degree about a certain axis. This may be interpreted by the control system 424 as a request to rotate the end effector 318 by 1 degree (if no rotational scaling is applied) about the corresponding axis.

In step S614 the control system 424 converts the matrix representation into an axis-angle representation of the rotation of the surgeon input device 423. As described above with reference to FIG. 8, the axis-angle representation of the rotation comprises: (i) an indication of a rotation axis for the rotation (e.g. an axis e), and (ii) an angle of rotation (e.g. θ) about the rotation axis. The rotation axis does not necessarily correspond to (e.g. align with) any particular physical component in the system. A person skilled in the art would know of methods for converting a 3D matrix representation of a rotation to an axis-angle representation of the rotation, and for conciseness the details of such methods are not included herein.

In step S616 the control system 424 scales the angle of rotation (θ) with a rotational scaling factor(s) to determine a scaled angle of rotation (θ′) about the rotation axis (e) for a scaled axis-angle representation of a scaled rotation, where θ′=sθ. The scaled axis-angle representation of the scaled rotation comprises: (i) an indication of the rotation axis (e) for the rotation, and (ii) the scaled angle of rotation (θ′) about the rotation axis. It is noted that the scaling does not alter the rotation axis (e).

In steps S618 and S620 the control system 424 generates a control signal for the surgical robot arm 301 using the scaled axis-angle representation of the scaled rotation. In particular, in step S618 the control system 424 converts the scaled axis-angle representation of the scaled rotation to a matrix representation of the scaled rotation. Again, a person skilled in the art would know of methods for converting an axis-angle representation of a rotation to a matrix representation of the rotation, and for conciseness the details of such methods are not included herein. Then in step S620 the control system 424 generates the control signal for the surgical robot arm 301 using the matrix representation of the scaled rotation.

The control system 424 is configured to generate control signals for the surgical robot arm 301 using matrix representations of rotations because this is how the control system 424 operates when the rotational scaling mode is not enabled. In particular, the control system 424 may generate the control signal for the surgical robot arm 301 by implementing inverse kinematics. A person skilled in the art would be aware of methods for generating a control signal by implementing inverse kinematics, and for conciseness the details of such methods are not included herein. The control signal indicates how the surgical robot arm 301 is to be driven in order to alter its configuration in accordance with the scaled movement of the surgeon input device 423.

In step S622 the control system 424 causes the generated control signal to be sent to the surgical robot arm in order to drive the surgical robot arm 301 such that its configuration is altered. In particular, the control signal instructs the surgical robot arm 301 how to move its joints (using the motors) in order to adopt the desired configuration in accordance with the scaled rotational motion of the surgeon input device 423.

Returning to FIG. 6a, if the control system 424 determines in step S608 that the user has not selected to enable the rotational scaling mode (e.g. that they have actively selected to disable the rotational scaling mode), then the method passes to step S624. For example, the control system 424 is configured to disable the rotational scaling mode in response to receiving a mode selection input that indicates that the user has selected to disable the rotational scaling mode.

Steps S624 to S630 are performed when the rotational scaling mode is disabled. In step S624 the control system 424 receives a control input from the surgeon input device 423. As described above, the control input indicates a requested movement of the surgical instrument 306.

In step S626 the control system 424 uses the received control input to determine an indication of a movement of the surgeon input device 423, e.g. by implementing forward kinematics. The indication of the movement comprises a matrix representation of a rotation of the surgeon input device 423. As described above, the matrix representation of the rotation of the surgeon input device 423 represents the rotation in the reference frame of the surgeon input device 423, thereby representing a three dimensional rotation in the coordinate system of the 3D reference frame of the surgeon input device 423. Since the rotational scaling mode is disabled, there is no need to convert the matrix representation into an axis-angle representation of the rotation of the surgeon input device 423.

In step S628 the control system 424 generates a control signal (herein referred to as a non-rotationally scaled control signal) for the surgical robot arm 301 using the determined indication of a movement of the surgeon input device 424, e.g. by implementing inverse kinematics. This is done without converting the matrix representation into an axis-angle representation of the rotation of the surgeon input device and without scaling the angle of rotation with a rotational scaling factor (because the rotational scaling mode is disabled). The control signal generated in step S628 indicates how the surgical robot arm 301 is to be driven in order to alter its configuration in accordance with the (non-scaled) movement of the surgeon input device 423.

In step S630 the control system 424 causes the generated non-rotationally scaled control signal to be sent to the surgical robot arm 301 in order to drive the surgical robot arm such that its configuration is altered. In particular, the non-rotationally scaled control signal instructs the surgical robot arm 301 how to move its joints in order to adopt the desired configuration in accordance with the non-scaled rotational motion of the surgeon input device 423.

In the examples described above, steps S602 and S604 are performed when the rotational scaling mode is not enabled and are for identifying that the rotational scaling mode may be useful and prompting the user to enable the rotational scaling mode. Similarly, when the rotational scaling mode is enabled the control system 424 may (in a step similar to step S602) identify a condition that suggests that the surgical robot arm 301 is not being used for a surgical step involving rotation of an instrument attached to the surgical robot arm. For example, the control system 424 may determine that none of the conditions mentioned above in relation to step S602 which suggest that the surgical robot arm 301 is being used for a surgical step involving rotation are present. For example, the control system 424 may determine that there are no needle holders attached to any of the surgical robot arms 301 in the surgical robotic system 400. In response to identifying the condition, the control system 424 may (in a step similar to step S604) output a prompt for prompting the user to disable the rotational scaling mode, e.g. on the display 421 and/or via the speaker 426 of the surgeon console 420. In response to the prompt, the user can make a selection (in any suitable manner) to disable the rotational scaling mode, such that the control system 424 receives a mode selection input in step S608 to indicate that the user has selected to disable the rotational scaling mode. The method then proceeds from step S608 to step S624 as described above.

In some examples, the user might be able to enable the rotational scaling mode only when a surgical instrument of a particular instrument type (e.g. a needle holder) is attached to the surgical robot arm 301. A surgical instrument 306 may have an RFID (Radio Frequency Identifier, or “tag”) and when it is attached to a surgical robot arm 301 a handshake procedure may be performed so that data can be passed between the surgical robot arm 301 and the surgical instrument 306. In particular, the surgical instrument 306 may comprise an instrument memory which can store some data relating to the surgical instrument (e.g. an instrument type, an instrument ID, a life count, etc.) and when the surgical instrument 306 is attached to the surgical robot arm 301 and the handshake procedure has been completed then the surgical robot arm 301 may be able to write data to and read data from the instrument memory on the surgical instrument 306. In this way, the surgical robot arm 301 can read the instrument type of the surgical instrument 306 and pass this information to the control system 424, and based on that information the control system 424 may determine whether to allow the rotational scaling mode to be enabled. In particular, the control system 424 may enable the rotational scaling mode and/or scale the angle of rotation (θ) in dependence upon the determined instrument type of the instrument. For example, the rotational scaling mode might only be enabled for controlling needle holders. In other words, the rotational scaling mode might only be enabled when a needle holder is attached to the surgical robot arm 301. This is because it is needle holders that typically need to be repeatedly rotated by approximately 270 degrees during a suturing step. Limiting the rotational scaling for use with certain types of instruments can avoid enabling the rotational scaling mode for instruments for which the rotational scaling mode is not suitable, and can also reduce the number of unnecessary prompts that are outputted in step S604 when rotational scaling is not desired.

There may be scaling options or limits that are specific to particular instrument types. Indications of these scaling options or limits may be stored in the instrument memory or in a system memory of the surgical robotic system 400.

In some examples, rather than providing prompts for quantitative rotational scaling factors (e.g. ‘1×’, ‘1.5×’, ‘2×’, etc.), the prompt output in step S604 may give the user qualitative rotational scaling options, e.g. “no scaling”, “medium scaling”, “high scaling”. In these examples, the control system 424 has freedom to choose the parameters (e.g. the rotational scaling factor) that are most suited for a given instrument to provide the rotational scaling that the surgeon may expect. This is another type of “instrument dependent” functionality that could be stored in the instrument memory. As an example, some instruments (e.g. some needle holders) make use of mechanical advantage to sacrifice jaw spread and yaw in exchange of greater grip force. For these instruments the rotational scaling may be less “aggressive”, i.e. a smaller rotational scaling factor may be applied.

In some situations the surgical robot arm 301 is not able to move exactly in accordance with the control input that is received from the surgeon input device 423 and the desired rotational scaling. This is because there are restrictions and/or limitations on the way in which the surgical robot arm can move, e.g. there may be a limit on the speed with which any particular part of the surgical robot arm 310 can move, there may be limits on the angles that can be adopted by each of the joints 304, or there may be an obstruction (e.g. another surgical robot arm in the surgical robotic system 400) physically preventing the surgical robot arm 301 from moving to the desired configuration. The movement of the surgical robot arm 301 can be measured (e.g. by the position sensors 307). For example, when the control system 424 has calculated a desired configuration of the surgical robot arm (e.g. by implementing inverse kinematics as described above) then it can determine whether the demanded movements for the individual joints of the surgical robot arm would exceed any of the limits. In particular, the control system 424 knows where the joints currently are and the additional motion that is demanded according to the control input received from the surgeon input device 423 and the desired rotational scaling. The system may determine the current position of the joints by means of the position sensors, or using the last demanded position (from the previous iteration). If the desired position of the surgical robot arm 301 (according to the instructed movement) would exceed a joint limit then the control system 424 may determine that the surgical robot arm 301 is not able to move exactly in accordance with the desired motion. As such, the control system 424 may determine that the surgical robot arm 301 cannot alter its configuration to achieve a desired configuration in accordance with a control signal that was generated using the scaled axis-angle representation of the scaled rotation. In response to this determination, the control system 424 may scale the angle of rotation with an adjusted rotational scaling factor to determine an adjusted scaled angle of rotation about the rotation axis for an adjusted scaled axis-angle representation of an adjusted scaled rotation, and then generate an adjusted control signal for the surgical robot arm 301 using the adjusted scaled axis-angle representation of the adjusted scaled rotation. The control system 424 can then cause the adjusted generated control signal to be sent to the surgical robot arm 301 in order to drive the surgical robot arm such that its configuration is altered. In this way it is only the magnitude of the angle of rotation that is adjusted (rather than adjusting the rotation axis). For example, the adjusted rotational scaling factor may be closer to 1 than the original rotational scaling factor.

The description above focusses on the rotational component of the movement of the surgeon input device 423. However, the indication of movement of the surgeon input device 423 which is determined in step S612 (or step S626) may also comprise a representation of a translational movement of the surgeon input device 423 (e.g. the hand controller 415). The control system 424 does not scale the translational movement of the surgeon input device 423 using the rotational scaling factor. The control system 424 may, or may not, scale the translational movement of the surgeon input device 423 using a translational scaling factor. The translational scaling factor may be different to the rotational scaling factor. If the control system 424 does scale the translation movement of the surgeon input device 423 using the translational scaling factor, then the control system 424 may generate the control signal in dependence on the scaled translational movement of the surgeon input device.

As described above, the rotational movement involved for suturing is approximately 270° at the end effector 318, but the surgeon's hand and/or the hand controller 415 is only capable of up to rotating by approximately 180° without having to use the clutch. Enabling the rotational scaling mode effectively increases the available range of motion for the surgeon in hand space, so they will have to disengage-reposition-reengage (i.e. use the clutch) less frequently. For example, using a rotational scaling factor of 1.5 or 2 may allow the surgeon to rotate the end effector by 270 degrees during suturing without using the clutch at all. Such a rotation may be performed many times during suturing.

Examples have been described above with reference to rotating a needle holder during suturing. In other examples, the surgical step could be something other than suturing that involves rotation of the instrument, e.g. super fine dissection, and/or the surgical instrument could be something other than a needle holder, e.g. a bladed instrument or an electrosurgical instrument which may be used to cut and seal tissue.

The methods described herein may be implemented by executing computer program code at the control system 424. That is, the control system 424 may comprise a computer readable storage medium having stored thereon computer readable instructions that, when executed on a processing unit at the control system 424, cause the control system to perform any of the methods described herein.

It is to be understood that the robot arm described herein could be for purposes other than surgery. For example, the surgical robot arm could be controlled for manipulating tissue, which is not part of a patient, e.g. for manipulating tissue of a cadaver or of any other object. As another example, the access port could be an inspection port in a manufactured article such as a car engine and the robot arm could control a viewing instrument for viewing inside the engine. The rotational scaling described herein could be particularly useful when controlling a viewing instrument (e.g. an endoscope) to allow the view to be rotated by more than the surgeon input device can be rotated without using the clutch.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A control system of a surgical robotic system, the surgical robotic system comprising a surgical robot arm and a surgeon input device, wherein the surgical robot arm comprises: (i) a series of joints by which its configuration can be altered, and (ii) an attachment for a surgical instrument at a distal end of the surgical robot arm, wherein the control system is configured to:
identify a condition that suggests that the surgical robot arm is being used for a surgical step involving rotation of a surgical instrument attached to the surgical robot arm; in response to identifying said condition, output a prompt for prompting a user to enable a rotational scaling mode;
receive a mode selection input that indicates whether the user has selected to enable the rotational scaling mode; and
enable the rotational scaling mode in response to receiving a mode selection input that indicates that the user has selected to enable the rotational scaling mode;
wherein the control system is configured to, when the rotational scaling mode is enabled:
receive a control input from the surgeon input device;
use the received control input to determine an indication of a movement of the surgeon input device, wherein the indication of the movement comprises a matrix representation of a rotation of the surgeon input device;
convert the matrix representation into an axis-angle representation of the rotation of the surgeon input device, wherein said axis-angle representation of the rotation comprises: (i) an indication of a rotation axis for the rotation, and (ii) an angle of rotation about the rotation axis;
scale the angle of rotation with a rotational scaling factor to determine a scaled angle of rotation about the rotation axis for a scaled axis-angle representation of a scaled rotation;
generate a control signal for the surgical robot arm using the scaled axis-angle representation of the scaled rotation; and
cause the generated control signal to be sent to the surgical robot arm in order to drive the surgical robot arm such that its configuration is altered.

2. The control system of claim 1 wherein the surgical robotic system comprises a surgeon console which comprises the surgeon input device, and wherein the control system is configured to output a prompt for prompting a user to enable a rotational scaling mode by:
causing a visual indication to be displayed at the surgeon console prompting the user to enable the rotational scaling mode, and/or
causing an audio indication to be output from the surgeon console prompting the user to enable the rotational scaling mode.

3. The control system of claim 1, wherein the control system is configured to receive the mode selection input responsive to the user making a selection on the surgeon console to enable the rotational scaling mode.

4. The control system of claim 3 wherein the selection is made by the user by:
selecting an option displayed on a display of the surgeon console;
issuing a voice command which is received by a microphone of the surgeon console; or
performing a gesture which is identified in images of the user captured by a camera of the surgeon console.

5. The control system of claim 1, wherein the mode selection input indicates the rotational scaling factor based on a user selection of the rotational scaling factor.

6. The control system of claim 1 further configured to determine an instrument type of a surgical instrument attached to the surgical robot arm, wherein the control system is configured to enable the rotational scaling mode and/or scale the angle of rotation in dependence upon the determined instrument type of the surgical instrument.

7. The control system of claim 6 wherein the control system is configured to determine the instrument type of the surgical instrument by reading an indication of the instrument type from an instrument memory located on the surgical instrument.

8. The control system of claim wherein the control system is configured to enable the rotational scaling mode only if the determined instrument type indicates that the surgical instrument is a needle holder.

9. The control system of claim 1 wherein said surgical step involving rotation of a surgical instrument attached to the surgical robot arm is suturing.

10. The control system of claim 9 wherein the control system is configured to identify the condition that suggests that the surgical robot arm is being used for suturing by:
determining that the surgical robotic system currently has one or more operational surgical robot arms with needle holder instruments attached thereto;
determining that the surgical robotic system currently has two or more operational surgical robot arms with a predetermined combination of surgical instruments attached thereto, said predetermined combination of surgical instruments being suitable for performing suturing;
determining that the current time in a surgical procedure matches a time at which suturing is planned to be performed in the surgical procedure;
analysing one or more images of a surgical site captured by an endoscope in the surgical robotic system; and/or
determining that a speed and/or pattern of motion of the surgical robot arm matches that expected during suturing.

11. The control system of claim 1 further configured to:
determine that the surgical robot arm cannot alter its configuration to achieve a desired configuration in accordance with the generated control signal using the scaled axis-angle representation of the scaled rotation;
in response to determining that the surgical robot arm cannot alter its configuration to achieve the desired configuration, scale the angle of rotation with an adjusted rotational scaling factor to determine an adjusted scaled angle of rotation about the rotation axis for an adjusted scaled axis-angle representation of an adjusted scaled rotation;
generate an adjusted control signal for the surgical robot arm using the adjusted scaled axis-angle representation of the adjusted scaled rotation; and
cause the adjusted generated control signal to be sent to the surgical robot arm in order to drive the surgical robot arm such that its configuration is altered.

12. The control system of claim 1 wherein the control system is configured to generate the control signal for the surgical robot arm using the scaled axis-angle representation of the scaled rotation by:
converting the scaled axis-angle representation of the scaled rotation to a matrix representation of the scaled rotation; and
generating the control signal for the surgical robot arm using the matrix representation of the scaled rotation.

13. The control system of claim 1 wherein the control system is configured to disable the rotational scaling mode in response to receiving a mode selection input that indicates that the user has selected to disable the rotational scaling mode, wherein the control system is configured to, when the rotational scaling mode is disabled:
receive a control input from the surgeon input device;
use the received control input to determine an indication of a movement of the surgeon input device, wherein the indication of the movement comprises a matrix representation of a rotation of the surgeon input device;
generate a non-rotationally scaled control signal for the surgical robot arm using the determined indication of a movement of the surgeon input device without converting the matrix representation into an axis-angle representation of the rotation of the surgeon input device and without scaling the angle of rotation with the rotational scaling factor; and
cause the generated non-rotationally scaled control signal to be sent to the surgical robot arm in order to drive the surgical robot arm.

14. The control system of claim 1 wherein the control system is configured to, when the rotational scaling mode is enabled:
identify a further condition that suggests that the surgical robot arm is not being used for a surgical step involving rotation of a surgical instrument attached to the surgical robot arm; and
in response to identifying said further condition, output a prompt for prompting the user to disable the rotational scaling mode.

15. The control system of claim 1 wherein the surgeon input device comprises a hand controller connected to a gimbal assembly, and wherein said received control input comprises indications of joint positions of the gimbal assembly.

16. The control system of claim 1 wherein the control system is configured to control the surgical robot arm, in dependence on the control inputs received from the surgeon input device, to alter the configuration of the surgical robot arm whilst maintaining an intersection between a surgical instrument attached to the surgical robot arm and a pivot point.

17. The control system of claim 1 wherein the indication of the movement of the surgeon input device further comprises a representation of a translational movement of the surgeon input device, wherein the control system is configured to:
scale the translational movement of the surgeon input device using a translational scaling factor and without using the rotational scaling factor; and
generate the control signal in dependence on the scaled translational movement of the surgeon input device.

18. A surgical robotic system comprising:
a surgical robot arm comprising a series of joints by which its configuration can be altered, the surgical robot arm having an attachment for a surgical instrument at a distal end of the surgical robot arm;
a surgeon input device; and
a control system as claimed in claim 1.

19. A method of controlling a surgical robot arm in a surgical robotic system, the surgical robotic system comprising the surgical robot arm and a surgeon input device, wherein the surgical robot arm comprises: (i) a series of joints by which its configuration can be altered, and (ii) an attachment for a surgical instrument at a distal end of the surgical robot arm, the method comprising:
identifying a condition that suggests that the surgical robot arm is being used for a surgical step involving rotation of a surgical instrument attached to the surgical robot arm;
in response to identifying said condition, outputting a prompt for prompting a user to enable a rotational scaling mode;
receiving a mode selection input that indicates that the user has selected to enable the rotational scaling mode;
enabling the rotational scaling mode in response to receiving the mode selection input; and
when the rotational scaling mode is enabled:
receiving a control input from the surgeon input device;
using the received control input to determine an indication of a movement of the surgeon input device, wherein the indication of the movement comprises a matrix representation of a rotation of the surgeon input device;
converting the matrix representation into an axis-angle representation of the rotation of the surgeon input device, wherein said axis-angle representation of the rotation comprises: (i) an indication of a rotation axis for the rotation, and (ii) an angle of rotation about the rotation axis;
scaling the angle of rotation with a rotational scaling factor to determine a scaled angle of rotation about the rotation axis for a scaled axis-angle representation of a scaled rotation;
generating a control signal for the surgical robot arm using the scaled axis-angle representation of the scaled rotation; and
causing the generated control signal to be sent to the surgical robot arm in order to drive the surgical robot arm such that its configuration is altered.

20. non-transitory computer readable storage medium having stored thereon computer readable instructions that, when executed at a control system for a surgical robotic system, cause the control system to perform the method of claim 19.
